(19) United States
(12) Patent Application Publication
(10) Pub. No.: US 2020/0342987 A1
(43) Pub. Date: Oct. 29, 2020

(54) System and Method for Information Exchange With a Mirror

(71) Applicant: doc.ai, Inc., Palo Alto, CA (US)

(72) Inventors: Walter Adolf DE BROUWER, Los Altos, CA (US); Srivatsa Akshay SHARMA, Palo Alto, CA (US); Scott Michael KIRK, Belmont, CA (US)

(73) Assignee: doc.ai, Inc., Palo Alto, CA (US)

(21) Appl. No.: 16/858,535

(22) Filed: Apr. 24, 2020

Related U.S. Application Data
(60) Provisional application No. 62/839,151, filed on Apr. 26, 2019.
(60) Provisional application No. 62/975,177, filed on Feb. 11, 2020.
(60) Provisional application No. 62/883,070, filed on Aug. 5, 2019.

(51) Int. Cl. G16H 40/67; G06N 5/04; G16H 30/20

(52) U.S. Cl. CPC G16H 40/67; G16H 30/20; G06N 5/04

(57) ABSTRACT

Method and system for remote medical information exchange are disclosed. The system for information exchange comprises a mirror configured to capture the facial information of a user and display inference information about the user. The system can comprise an image capture module configured to capture the facial information of the user while the user is looking into the mirror. The system can include a displaying module, coupled to a display integrated into the mirror and configured to cause display of inference information about the user. An on-mirror computation device, coupled in communication with the image capture module and the displaying module, is configured to receive and process facial information of the user and produce inference information about the user. The on-mirror computation device can comprise one or more artificial intelligence modules.

### PRIORITY APPLICATIONS

This application claims priority to or the benefit of U.S. Provisional Patent Application No. 62/839,151 titled, “SYSTEM AND METHOD FOR INFORMATION EXCHANGE WITH A MIRROR,” filed Apr. 26, 2019, (Attorney Docket No. DCAI 1009-1); U.S. Provisional Patent Application No. 62/975,177, titled, “ARTIFICIAL INTELLIGENCE-BASED DRUG ADHERENCE MANAGEMENT AND PHARMACOVIGILANCE,” filed Feb. 11, 2020, (Attorney Docket No. DCAI 1005-1); US Provisional Patent Application Nos. 62/883,070 titled, “ACCELERATED PROCESSING OF GENOMIC DATA AND STREAMLINED VISUALIZATION OF GENOMIC INSIGHTS,” filed Aug. 5, 2019, (Attorney Docket No. DCAI 1000-1).

### INCORPORATIONS

The following materials are incorporated by reference as if fully set forth herein:

U.S. patent application Ser. No. 16/816,153, titled “SYSTEM AND METHOD WITH FEDERATED LEARNING MODEL FOR MEDICAL RESEARCH APPLICATIONS,” filed on Mar. 11, 2020 (Atty. Docket No. DCAI 1008-2);

U.S. patent application Ser. No. 16/802,485, titled, “SYSTEM AND METHOD FOR REMOTE MEDICAL INFORMATION EXCHANGE,” filed Feb. 26, 2020 (Attorney Docket No. DCAI 1007-2).

### TECHNICAL FIELD

The disclosed system and method are in the field of smart electronic devices for information exchange, specifically in the field of smart mirror for medical information exchange and communication.

### BACKGROUND

In recent years, people are getting more and more educated about personal health care and medical information. Statistically, on average, a person spends around five hours a week in front of a mirror, grooming, checking oneself, or even pondering. In this era of information and data, this chunk of time is sunk time in terms of information and data collection, as there is no information captured for the end user.

People on the other hand spend a great amount of time using their smartphones. Generations with smartphones, which is almost all population in developed countries and growing population in developing countries, are enabled to check, collect, gather medical and health care information and data like never before. Smartphone with cameras allow people to take pictures of themselves, such as selfie images. Smartphone cameras also allow users to make video clips of themselves. The selfie images or images extracted from videos captured from cameras can be processed to provide health related information to users. However, the selfie images captured from the smartphone cameras are often taken at a very close distance to the user as the user is holding the camera in front of her face. The software applications process the images to extract features from the captured images. The selfie images often do not provide a complete picture of a user. This limitation often results in incorrect results generated from image processing systems. For example, a selfie image taken at a close distance to a user's face can result in an inaccurate prediction about the height of the user, which is an important input to various health related measurements such as Body Mass Index (BMI).

Therefore, an opportunity arises to capture information of a user when the user is standing in front of a mirror and utilize this information for better medical and health related predictions.

### SUMMARY

The technology disclosed is related to a smart mirror that can be used as a medical or health tracking device. The smart mirror, also referred to as a phenomenal mirror with which a person can track his/her phenotypic information on an ongoing basis is disclosed. The phenotypic information can include but is not limited to age, height, weight, mood, behavioral-health, life-expectancy, health-screening-plan, etc. The phenomenal mirror can have a complete view of a person when he/she stands in front of the mirror, facing the mirror. The mirror can generate multiple physiological parameter inference about the person.

Generally provided are a system and method for information exchange. The system can comprise a mirror which is configured to capture the facial information of a user and display inference information about the user. The system can comprise an image capture component, configured to capture facial information of the user. The system can include a display component, configured to augment and display inference information about the user. The system can include an on-mirror computation device, coupled with the mirror and configured to process facial information of the user. The on-mirror computation device can comprise one or more artificial intelligence components.

The system includes a phenomenal mirror. The phenomenal mirror is configured to capture the phenotypic information of a user when the user is facing the mirror. The system is configured to augment and display the inference from captured image(s) of the user back to the user as a display layered on top of the mirror for easy reading. In one embodiment, the entire mirror can be a digital display, enabled to present the phenotypic information to the user. In another embodiment, a portion of the mirror can include a display for presenting the phenotypic information to the user. The system is further configured to keep track of historical information of the user and provide quick historical summary and comparison of the person's health over time. This feature is also referred to as arrow of time of health and is readily available for health-conscious users. The system is further configured to keep track of individual profiles in a household to differentiate users using the same mirror. The system is further configured to comprise an image capture component such as a high definition camera, that captures the information of the user. The system can include logic to process images and perform computations of user information using on-device processing unit.

This summary is provided to efficiently present the general concept of the invention and should not be interpreted as limiting the scope of the claims.

### BRIEF DESCRIPTION OF THE DRAWINGS

For purpose of facilitating understanding of the embodiments, the accompanying drawings and description illustrate embodiments thereof, its various structures, construction, method of operation, and many advantages that may be understood and appreciated. According to common practice, the various features of the drawings are not drawn to scale. To the contrary, the dimensions of the various features are expanded or reduced for the purpose of explanation and clarity.

FIG. 1A is a process flowchart illustrating an example interaction session between an end user and the phenomenal mirror, consistent with embodiments of the present disclosure.

FIG. 1B presents an example screen display of the phenomenal mirror using the interaction session flowchart described in FIG. 1A, consistent with embodiments of the present disclosure.

FIG. 2A is a flowchart illustrating an example interaction session between an end user and the phenomenal mirror, consistent with embodiments of the present disclosure.

FIG. 2B presents an example screen display of the phenomenal mirror using the interaction session flowchart presented in FIG. 2A, consistent with embodiments of the present disclosure.

FIG. 2C presents another example screen display of the phenomenal mirror, consistent with embodiments of the present disclosure.

FIG. 3 presents an example architecture of a data storage center with cloud services and machine learning components, as well as the interaction between image data with these machine learning components, consistent with embodiments of the present disclosure.

FIG. 4 presents an example system architecture of data storage center with cloud services, consistent with embodiments of the present disclosure.

FIG. 5 is an example convolutional neural network.

FIG. 6 a block diagram illustrating training of the convolutional neural network of FIG. 5.

FIG. 7 is a simplified block diagram of a computer system that can be used to implement the technology disclosed.

### DETAILED DESCRIPTION

Many alternative embodiments of the technology disclosed may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more embodiments may also be included in combination with another embodiment even though not expressly shown and described in that specific combination.

For purpose of efficiency, reference numbers may be repeated between figures where they are intended to represent similar features between otherwise varied embodiments, though those features may also incorporate certain differences between embodiments and to the extent specified as such or otherwise apparent to one of ordinary skill, such as differences clearly shown between them in the respective figures.

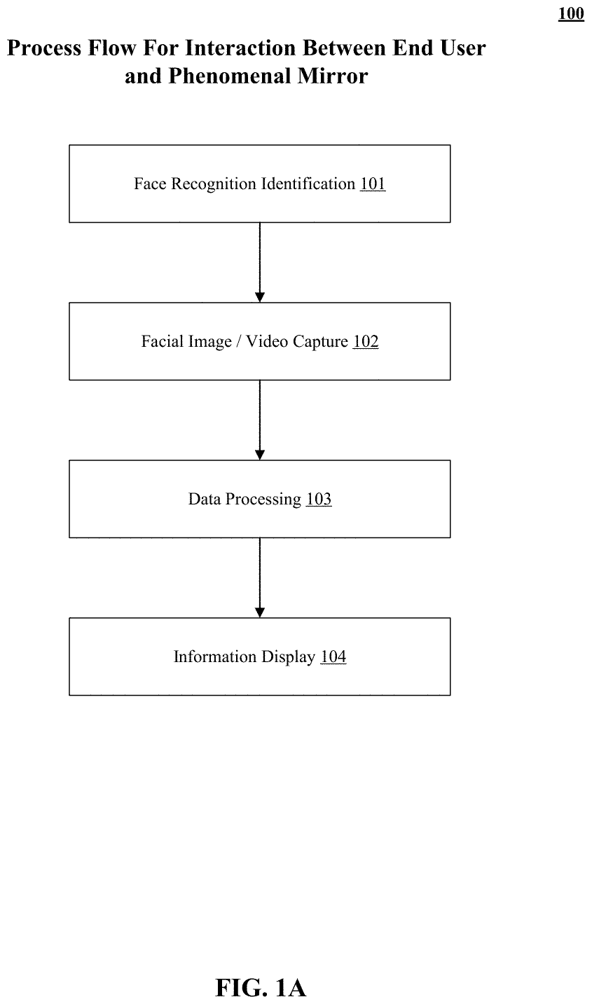

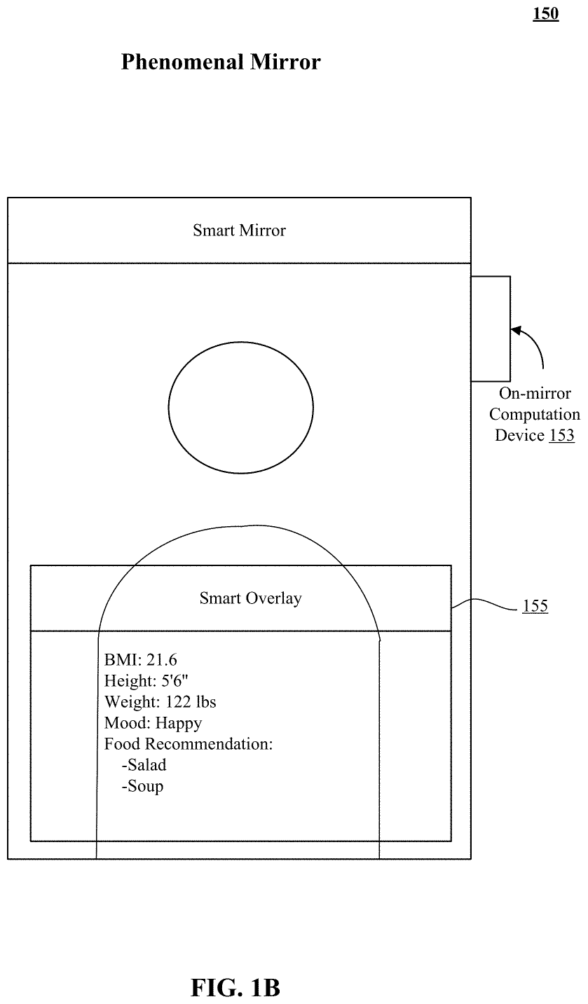

Reference is now made to FIG. 1A, which is an example flowchart 100 of an interaction session between an end user and the phenomenal mirror, consistent with embodiments of the present disclosure. The interaction session comprises four steps, namely step 101 face region identification, step 102 facial image capturing, step 103 data processing, and step 104 information displaying.

In step 101 face region identification, end user stands in front of a phenomenal mirror, and the phenomenal mirror identifies and zooms in to the face region. Facial features are the features of interest for phenotypic information. In one embodiment, the system can ignore the image of rest of the body of the end user. In another embodiment, the system can capture image of the entire body of the end user and process the full body image in the following steps or process selected portions of the end user's image. In step 102 facial image capturing, the end user starts to groom or do whatever he/she is pleased to do in front of the phenomenal mirror. Edge module installed in the phenomenal mirror starts to capture the end user's facial information and continue to process the phenotypic information. In step 103 data processing, captured facial information, which can be facial image and/or facial video stream, is processed and analyzed by a machine learning algorithm stored in an on-mirror computation device. In step 104 information displaying, augmented and layered information is displayed by a display module of the phenomenal mirror on the mirror to the user. In some embodiments, the phenomenal is configured to comprise a touch screen module to take inputs from end users. In some embodiments, the augmented information displayed back to the user will include all phenotypic information can include auto inference of age, sex, height, and weight, auto inference of mood and sentiments, auto inference of recommended health screenings and diagnostic information, etc.

In some embodiments, end user's facial data never leaves the mirror. It provides a significant guarantee of user's privacy. Hence end user can feel comfortable standing in front of the phenomenal mirror however he/she pleases. For the basic phenotypic 5 information the phenomenal mirror does not require internet connection to assure the end user that no information will be sent anywhere.

Reference is now made to FIG. 1B, which illustrates an example screen display of the phenomenal mirror (or smart mirror) 150 using the interaction session flowchart described in FIG. 1A, consistent with embodiments of the present disclosure.

With this example screen display of the phenomenal mirror, the end user continues to see the image of himself/herself in the mirror. On top of that, a smart overlay 155 is displayed to deliver derived information on the end user's physiological information and health related data, e.g., height, weight, BMI, mood, or some lifestyle information, e.g., food recommendation, etc. The information overlay can be augmented text, so that end user can read from a normal distance when he/she does his/her daily routines. The information overlay can also be designed that the background is transparent, so that the overlay information does not block the vision of end user. In one embodiment, the smart overlay 155 can cover the entire mirror surface. In another embodiment, the smart overlay can be positioned in the top portion of the phenomenal mirror. In another embodiment, the smart overlay can be positioned in one corner of the phenomenal mirror. In such an embodiment, the size of the smart overlay can be equivalent to the size of a hand-held device or a tablet. The phenomenal mirror includes an on-mirror computation device 153. We present further details of the on-mirror computation device in the following text.

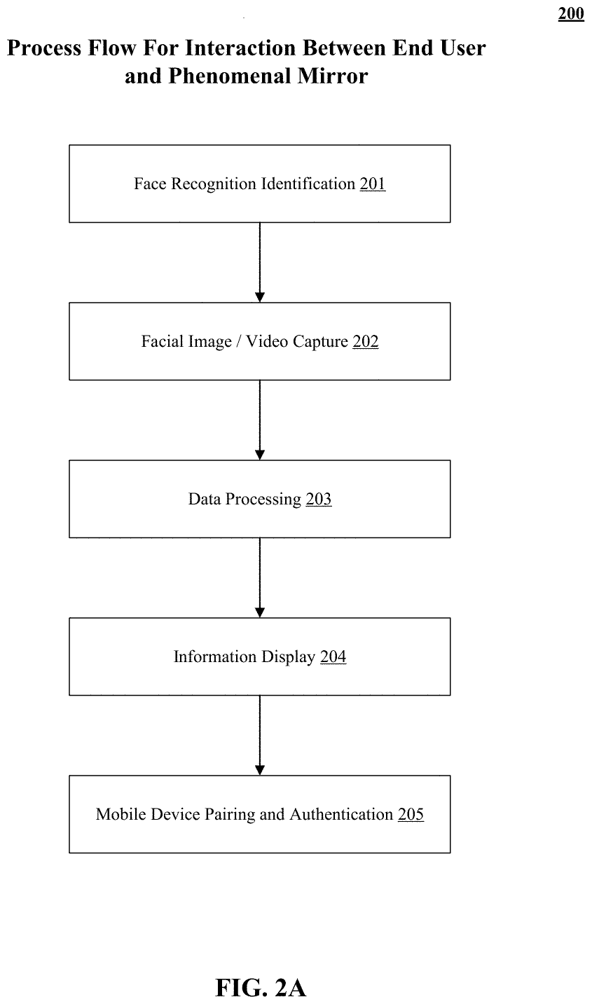

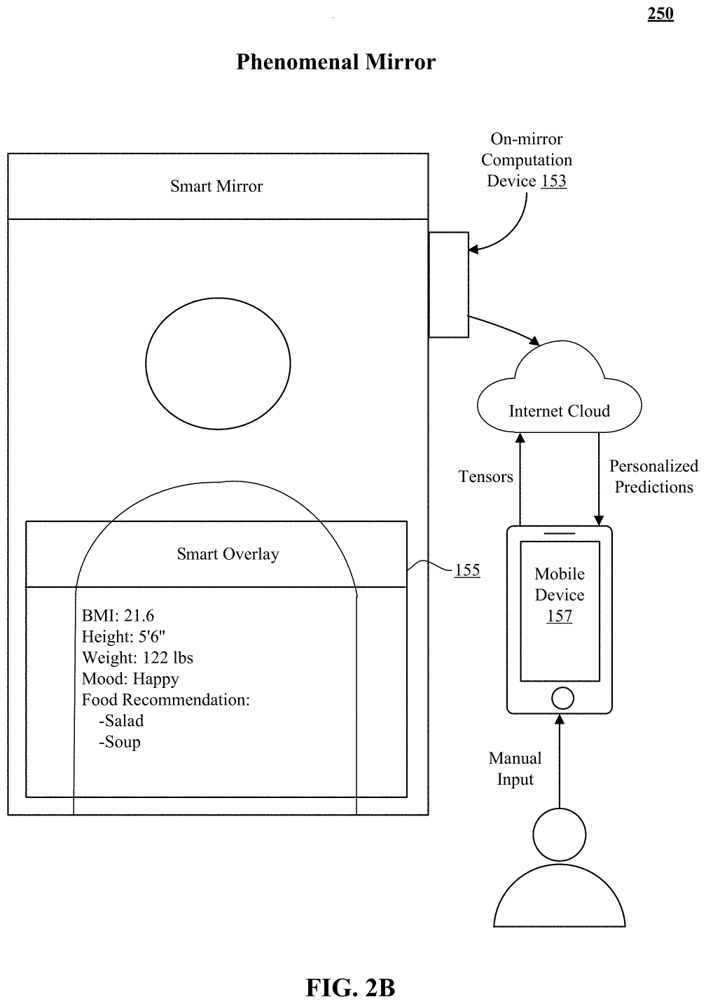

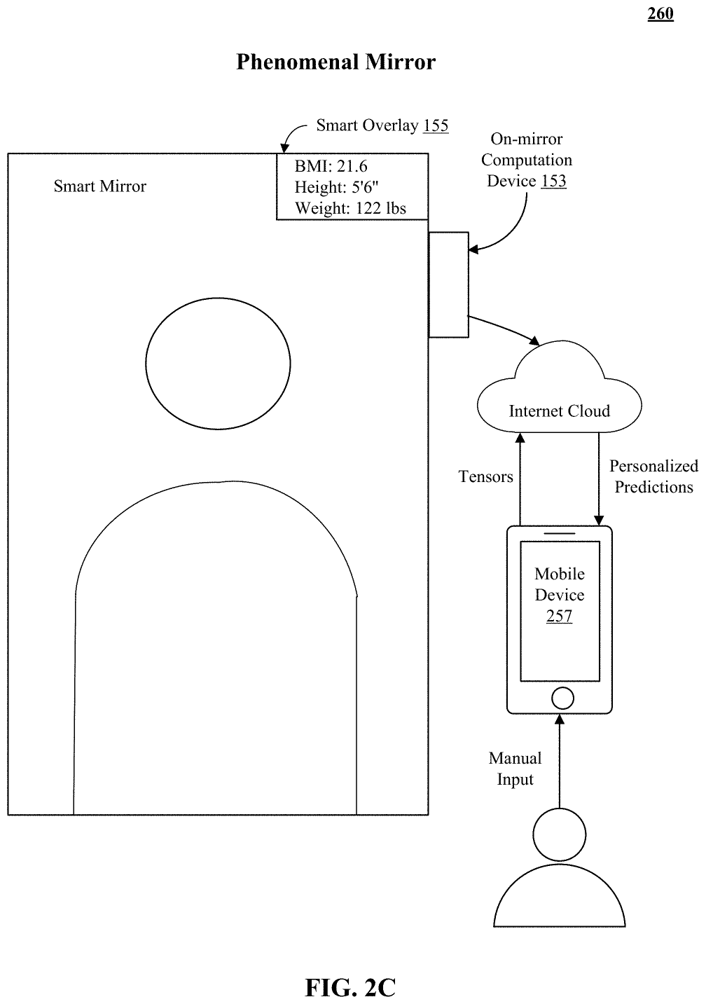

Reference is now made to FIG. 2A, which is a flowchart 200 illustrating an example interaction session between end user and the phenomenal mirror, consistent with embodiments of the present disclosure.

In addition to steps 201, 202, 203, and 204, which are identical to steps 101, 102, 103, and 104, the process includes a step 205 mobile device paring and authentication, to allow end user to connect to a local intranet network connection or similar, e.g., WIFI connection. A pairing between the phenomenal mirror and an application program of end user's mobile device, e.g., smart phone, tablet, etc., to authenticate the communication. In some embodiments, the application program preinstalled at the end user's mobile device is connected to additional information of the end user. Therefore, data or information about the end user can be further augmented and displayed in a more personalized manner.

Such additional information may comprise auto inference of exposome data like air quality, pollen exposure, socio-economic risk, or other relevant information based on the location from where the end user is connected. Such additional information may further comprise auto inference of risk for genetic diseases in the scenario that the end user has uploaded his/her genetic data via the application program to the mobile device. Such additional information may further comprise auto suggestion and tracking information on activities collected by tracking device via the application program in the mobile device. Such additional information may further comprise auto inference of past medical records and past prescriptions collected by camera via the application program in the mobile device.

Reference is now made to FIG. 2B (referred by a numeral 250), which presents an example screen display of the phenomenal mirror using the interaction session flowchart described in FIG. 2A, consistent with embodiments of the present disclosure.

In some embodiments, the on-mirror computation device 153 connected to the smart mirror comprises a transceiver communication part configured to connect to a cloud-based server or cloud-based storage. Based on preset configuration, the on-mirror computation device 153 is capable of sending and receiving health related information to and from the cloud-based server or the cloud-based storage. The cloud-based server or the cloud-based storage is further configured to connect to the end user's mobile device 257 in which a health-related application program is installed. The health-related application program is configured to collect personalized health data for the end user or from other third-party platform with the permission of the end user.

In some embodiments, the federated learning technologies are applied in the configuration. The mobile device is configured to push tensors based on personal health data of the end user to cloud-based server, without private and personal data leaving the mobile device. The cloud-based server is configured to update machine learning algorithm based on the received tensors and send back personalized predictions to the end user. Further details of the federated learning technologies are presented in our application. Further description of federated learning technologies is presented in SYSTEM AND METHOD WITH FEDERATED LEARNING MODEL FOR MEDICAL RESEARCH APPLICATIONS, incorporated above by reference.

FIG. 2C (referred to by a numeral 260) presents another embodiment of phenomenal mirror (or smart mirror) in which the smart overlay 155 is positioned on one corner of the mirror. In this embodiment, the smart overlay can present part of the phenotype information at one time. For example, the smart overlay 155 in FIG. 2C displays the BMI, height and weight information to the end user. The smart overlay can periodically change the phenotype information on the display to show additional phenotype information to the end user. It is understood that the number and type of phenotype information data shown on the display can be changed. The system can include user interface controls that allow the end user to scroll through the phenotype information as desired.

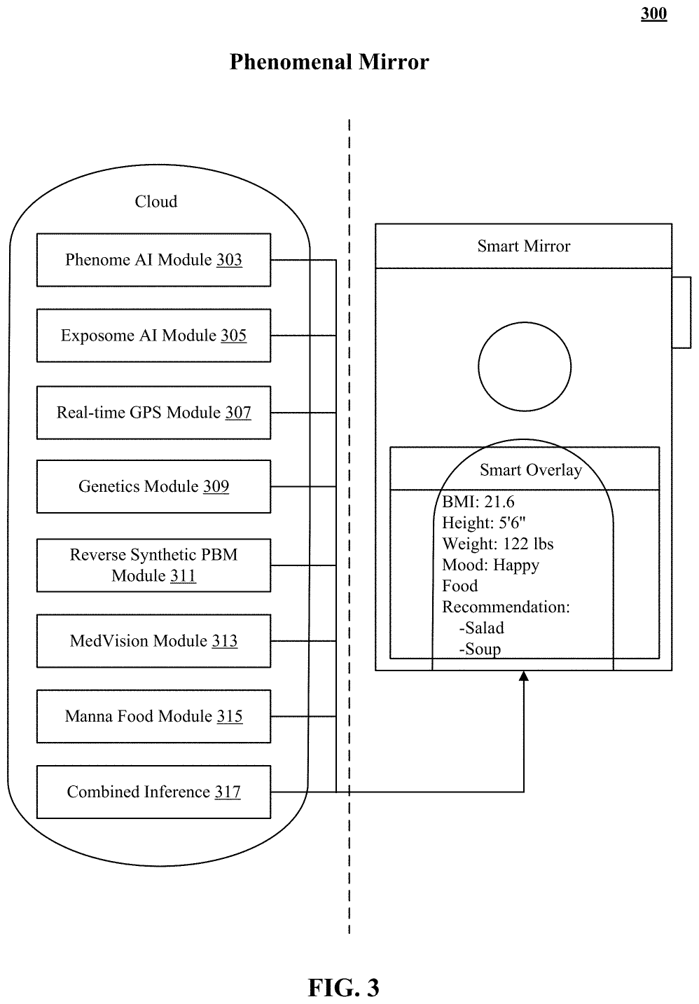

Reference is now made to FIG. 3, which is a diagram 300 illustrating an example data storage centre with cloud services and its machine learning modules, as well as the interaction between image data with these machine learning modules, consistent with embodiments of the present disclosure.

When the image data is streamed up to data storage centre with cloud services, machine learning modules pre-installed in the cloud start analyzing the image data. In some embodiments, machine learning module can include but are not limited to phenome artificial intelligence module 303, exposome artificial intelligence module 305, real-time Global Position System (GPS) or navigation module 307, genetics and bioinformatics module 309, reverse synthetic Pharmacy Benefit Manager (PBM) module 311, MedVision module 313, Manna food module 315, combined inference module 317, etc. The pre-installed machine learning modules can be one or more of any combination of the aforementioned modules. Each machine learning module functions as its name indicates.

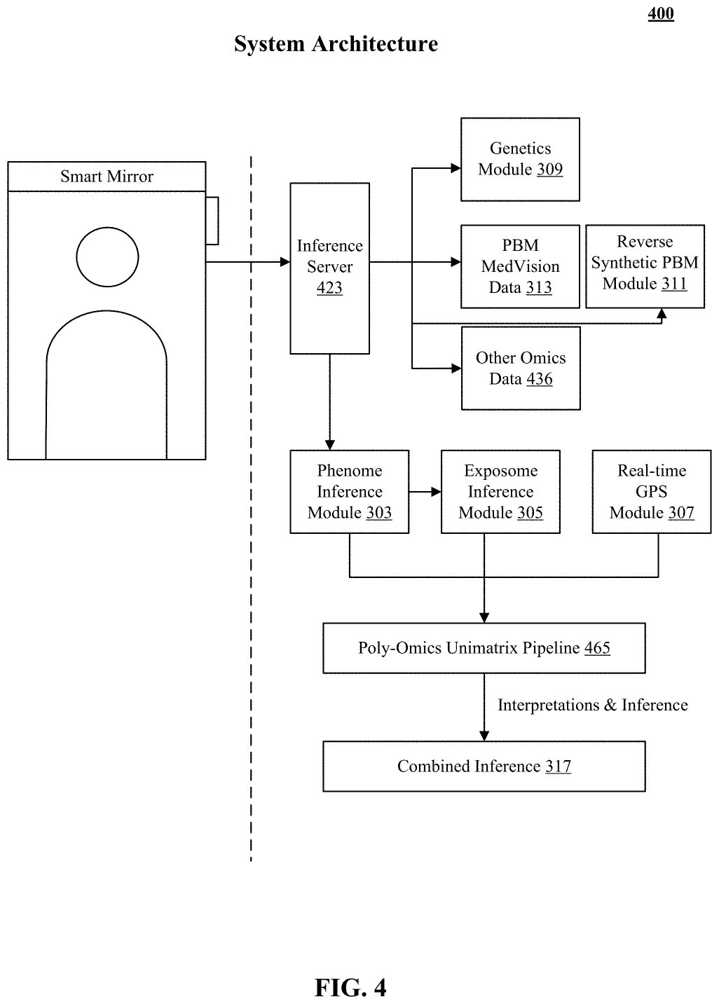

Reference is now made to FIG. 4, which is a diagram illustrating an example system architecture 400 of data storage centre with cloud-based services, consistent with embodiments of the present disclosure.

The system is largely a composable set of backend artificial intelligence-based modules that uses data from software development kit to make one or more predictions for real-time feedback. In some embodiments, the system can be configured to comprise inference server module 423, phenome artificial intelligence inference module 303, real-time GPS module 307, exposome artificial intelligence inference module 305, genetics and bioinformatics module 309, reverse synthetic PBM module 311, MedVision module 313, Manna food module 315, polyomics uni-matrix pipeline 465, and combined inference module 317.

Inference server module 423 is configured to receive shared edge inference, usually referred to as tensors, without requiring image data of the end user, to protect end user data privacy. Phenome artificial intelligence inference module 303 is configured to extract data and infers physiological parameters including age, sex, height, weight, BMI, mood, life-expectancy, or other statistical measures. Real-time GPS module 307 is configured to constantly synchronize to end user's physical location and past locations in order to understand the exposimics of the user. Exposome artificial intelligence inference module 305 is configured to compute the risks and exposures to air quality, pollen, walkability (how active one is allowed in the condition), socio-economic risks, etc. Inference data from this module augments the risk for certain medical conditions, e.g., Asthma, allergies, etc. Further description of these modules is found in ARTIFICIAL INTELLIGENCE-BASED DRUG ADHERENCE MANAGEMENT AND PHARMACOVIGILANCE, incorporated above by reference.

Genetics and bioinformatics module 309 is configured to allow an end user upload his/her genetic profile data, from consumer faced genetic services such as 23andMe™, Ancestry™, full exome sequence, whole genome sequence, etc., as part of the application program onboarding procedure. Further description of handling and analysis of genetic information if found in ACCELERATED PROCESSING OF GENOMIC DATA AND STREAMLINED VISUALIZATION OF GENOMIC INSIGHTS and ARTIFICIAL INTELLIGENCE-BASED DRUG ADHERENCE MANAGEMENT AND PHARMACOVIGILANCE, incorporated above by reference. The information collected by genetic and bioinformatics module can be used along with the combination of other phenotypic data from phenome artificial intelligence inference module 303. Reverse synthetic PBM module 311 is configured to allow end user to optionally add medications and prescription information using the MedVision module 313. Further description of PBM and MedVision modules is found in ARTIFICIAL INTELLIGENCE-BASED DRUG ADHERENCE MANAGEMENT AND PHARMACOVIGILANCE, incorporated above by reference. The reverse synthetic PBM module 311 curates the drug information for end user. Such drug information can also be cross-referenced with genetics data to red flag risks of certain drugs to end user.

MedVision module 313 is configured to allow end user to import his/her medical records and medication information by using the camera of his/her mobile device. End user can use the camera of a mobile device to take a picture of medications he/she takes. MedVision module 313 is configured to process the image to capture the medication information. Manna food module 315 is configured to estimate the probability that food in a picture is scientifically considered to be healthy or not. Poly-omics uni-matrix pipeline 465 is configured to be fed by data from some or all aforementioned artificial intelligence modules. It joins multiple omics collected to build predictive values on the end user in a real-time manner. Further description of polyomics is found in ARTIFICIAL INTELLIGENCE-BASED DRUG ADHERENCE MANAGEMENT AND PHARMACOVIGILANCE, incorporated above by reference. Combined inference module 317 is configured to combine all the predictions and inferences from some or all modules. Display feed data formed by combined inference module is to be passed back to end user and displayed on the phenomenal mirror.

Although the invention is illustrated and described herein with reference to specific embodiments, the invention is not intended to be limited to the details shown. Rather, various modifications can be made in the details within the scope of equivalents of the claims by anyone skill in the art without departing from the invention.

### Example Machine Learning Model

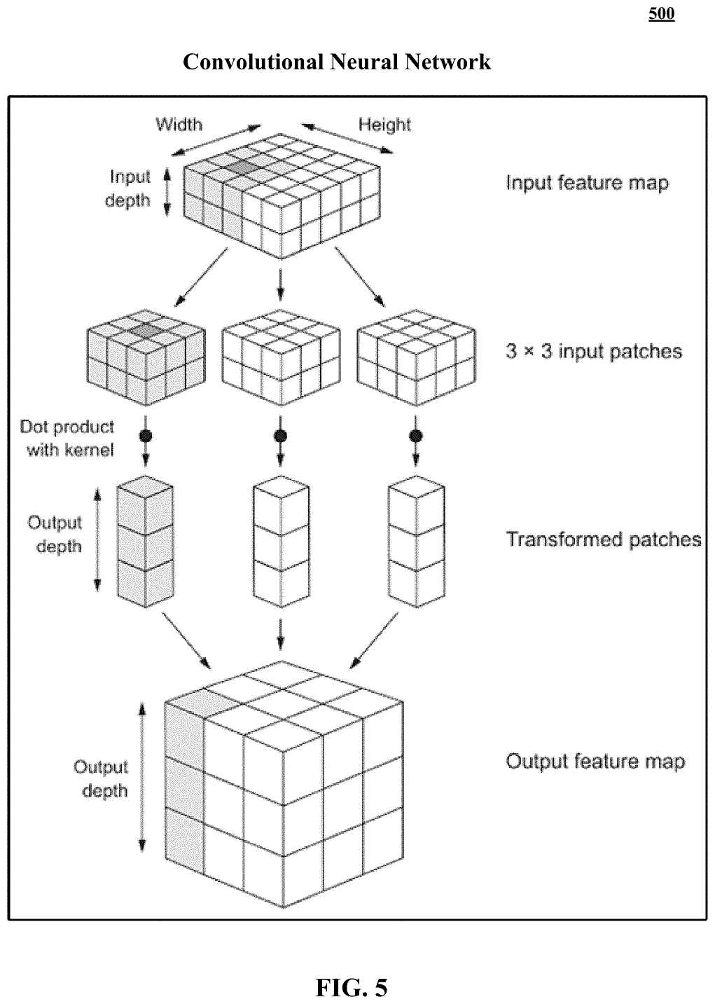

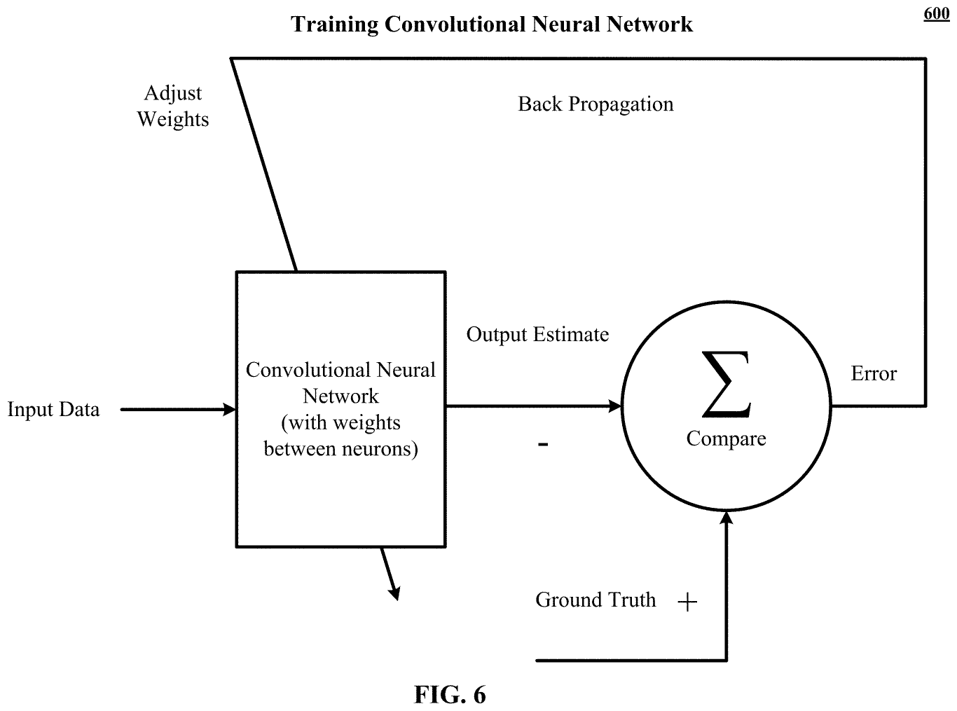

With this example in mind, we present an example machine learning model that can be used to process images from the phenomenal mirror. A general discussion of regarding convolutional neural networks, CNNs, and training by gradient descent is facilitated by FIGS. 5 and 6.

### Convolutional Neural Networks

A convolutional neural network 500 is a special type of neural network. The fundamental difference between a densely connected layer and a convolution layer is this: Dense layers learn global patterns in their input feature space, whereas convolution layers learn local patters: in the case of images, patterns found in small 2D windows of the inputs. This key characteristic gives convolutional neural networks two interesting properties: (1) the patterns they learn are translation invariant and (2) they can learn spatial hierarchies of patterns.

Regarding the first, after learning a certain pattern in the lower-right corner of a picture, a convolution layer can recognize it anywhere: for example, in the upper-left corner. A densely connected network would have to learn the pattern anew if it appeared at a new location. This makes convolutional neural networks data efficient because they need fewer training samples to learn representations they have generalization power.

Regarding the second, a first convolution layer can learn small local patterns such as edges, a second convolution layer will learn larger patterns made of the features of the first layers, and so on. This allows convolutional neural networks to efficiently learn increasingly complex and abstract visual concepts.

A convolutional neural network learns highly non-linear mappings by interconnecting layers of artificial neurons arranged in many different layers with activation functions that make the layers dependent. It includes one or more convolutional layers, interspersed with one or more sub-sampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enters the network, the convolutional neural network avoids the complexity of data reconstruction in feature extraction and regression or classification process.

Convolutions operate over 3D tensors, called feature maps, with two spatial axes (height and width) as well as a depth axis (also called the channels axis). For an RGB image, the dimension of the depth axis is 3, because the image has three color channels; red, green, and blue. For a black-and-white picture, the depth is 1 (levels of gray). The convolution operation extracts patches from its input feature map and applies the same transformation to all of these patches, producing an output feature map. This output feature map is still a 3D tensor: it has a width and a height. Its depth can be arbitrary, because the output depth is a parameter of the layer, and the different channels in that depth axis no longer stand for specific colors as in RGB input; rather, they stand for filters. Filters encode specific aspects of the input data: at a height level, a single filter could encode the concept “presence of a face in the input,” for instance.

For example, the first convolution layer takes a feature map of size (28, 28, 1) and outputs a feature map of size (26, 26, 32): it computes 32 filters over its input. Each of these 32 output channels contains a 26×26 grid of values, which is a response map of the filter over the input, indicating the response of that filter pattern at different locations in the input. That is what the term feature map means: every dimension in the depth axis is a feature (or filter), and the 2D tensor output [:, :, n] is the 2D spatial map of the response of this filter over the input.

Convolutions are defined by two key parameters: (1) size of the patches extracted from the inputs—these are typically 1×1, 3×3 or 5×5 and (2) depth of the output feature map—the number of filters computed by the convolution. Often these start with a depth of 32, continue to a depth of 64, and terminate with a depth of 128 or 256.

A convolution works by sliding these windows of size 3×3 or 5×5 over the 3D input feature map, stopping at every location, and extracting the 3D patch of surrounding features (shape (window_height, window width, input_depth)). Each such 3D patch is ten transformed (via a tensor product with the same learned weight matrix, called the convolution kernel) into a 1D vector of shape (output_depth,). All of these vectors are then spatially reassembled into a 3D output map of shape (height, width, output_depth). Every spatial location in the output feature map corresponds to the same location in the input feature map (for example, the lower-right corner of the output contains information about the lower-right corner of the input). For instance, with 3×3 windows, the vector output [i, j, :] comes from the 3D patch input [i−1: i+1, j−1:J+1, :].

The convolutional neural network comprises convolution layers which perform the convolution operation between the input values and convolution filters (matrix of weights) that are learned over many gradient update iterations during the training. Let (m, n) be the filter size and W be the matrix of weights, then a convolution layer performs a convolution of the W with the input X by calculating the dot product W·x+b, where x is an instance of X and b is the bias. The step size by which the convolution filters slide across the input is called the stride, and the filter area (m×n) is called the receptive field. A same convolution filter is applied across different positions of the input, which reduces the number of weights learned. It also allows location invariant learning, i.e., if an important pattern exists in the input, the convolution filters learn it no matter where it is in the sequence.

### Training a Convolutional Neural Network

FIG. 6 depicts a block diagram 600 of training a convolutional neural network in accordance with one implementation of the technology disclosed. The convolutional neural network is adjusted or trained so that the input data leads to a specific output estimate. The convolutional neural network is adjusted using back propagation based on a comparison of the output estimate and the ground truth until the output estimate progressively matches or approaches the ground truth.

The convolutional neural network is trained by adjusting the weights between the neurons based on the difference between the ground truth and the actual output. This is mathematically described as:

$$Δ {w}_{i} = {x}_{i} δ$$

$$where δ = ( ground truth ) - ( actual output )$$

In one implementation, the training rule is defined as:

wₙₘ→wₙₘ+α(tₘ−φₘ)aₙ

In the equation above: the arrow indicates an update of the value; tₘ is the target value of neuron m; φₘ is the computed current output of neuron m; an aₙ is input n; and α is the learning rate.

The intermediary step in the training includes generating a feature vector from the input data using the convolution layers. The gradient with respect to the weights in each layer, starting at the output, is calculated. This is referred to as the backward pass, or going backwards. The weights in the network are updated using a combination of the negative gradient and previous weights.

In one implementation, the convolutional neural network uses a stochastic gradient update algorithm (such as ADAM) that performs backward propagation of errors by means of gradient descent. One example of a sigmoid function based back propagation algorithm is described below:

$$ϕ = f ( h ) = \frac{1}{1 + {e}^{- h}}$$

In the sigmoid function above, h is the weighted sum computed by a neuron. The sigmoid function has the following derivative:

$$\frac{\partial ϕ}{\partial h} = ϕ ( 1 - ϕ )$$

The algorithm includes computing the activation of all neurons in the network, yielding an output for the forward pass. The activation of neuron m in the hidden layers is described as:

$${ϕ}_{m} = \frac{1}{1 + {e}^{- hm}}$$

$${h}_{m} = {\sum }_{n = 1}^{N} {a}_{n} {w}_{nm}$$

This is done for all the hidden layers to get the activation described as:

$${ϕ}_{k} = \frac{1}{1 + {e}^{hk}}$$

$${h}_{k} = {\sum }_{m = 1}^{M} {ϕ}_{m} {v}_{mk}$$

Then, the error and the correct weights are calculated per layer. The error at the output is computed as:

δₒₖ=(tₖ−φₖ)φₖ(1−φₖ)

The error in the hidden layers is calculated as:

$${δ}_{hm} = {ϕ}_{m} ( 1 - {ϕ}_{m} ) {\sum }_{k = 1}^{K} {v}_{mk} {δ}_{ok}$$

The weights of the output layer are updated as:

vₘₖ→vₘₖ+αδₒₖφₘ

The weights of the hidden layers are updated using the learning rate α as:

vₙₘ→vₙₘ+αδₕₘaₙ

In one implementation, the convolutional neural network uses a gradient descent optimization to compute the error across all the layers. In such an optimization, for an input feature vector x and the predicted output ŷ, the loss function is defined as l for the cost of predicting ŷ when the target is y, i.e. l (ŷ, y). The predicted output ŷ is transformed from the input feature vector x using function ƒ. Function ƒ is parameterized by the weights of convolutional neural network, i.e. ŷ=ƒ_{w}(x). The loss function is described as l (ŷ, y)=l (ƒ_{w} (x), y), or

Q(z, w)=l (ƒ_{w} (x), y) where z is an input and output data pair (x, y). The gradient descent optimization is performed by updating the weights according to:

$${v}_{t + 1} = μ {v}_{t} - α \frac{1}{n} {\sum }_{i = 1}^{N} ∇ {w}_{t} Q ( {z}_{t} , {w}_{t} )$$

$${w}_{t + 1} = {w}_{t} + {v}_{t + 1}$$

In the equations above, α is the learning rate. Also, the loss is computed as the average over a set of n data pairs. The computation is terminated when the learning rate α is small enough upon linear convergence. In other implementations, the gradient is calculated using only selected data pairs fed to a Nesterov's accelerated gradient and an adaptive gradient to inject computation efficiency.

In one implementation, the convolutional neural network uses a stochastic gradient descent (SGD) to calculate the cost function. A SGD approximates the gradient with respect to the weights in the loss function by computing it from only one, randomized, data pair, Zₜ, described as:

vₜ₊₁=μv−α□wQ(zₜ,wₜ)

wₜ₊₁=wₜ+vₜ₊₁

In the equations above: α is the learning rate; μ is the momentum; and t is the current weight state before updating. The convergence speed of SGD is approximately O(1/t) when the learning rate α are reduced both fast and slow enough. In other implementations, the convolutional neural network uses different loss functions such as Euclidean loss and softmax loss. In a further implementation, an Adam stochastic optimizer is used by the convolutional neural network.

### Computer System

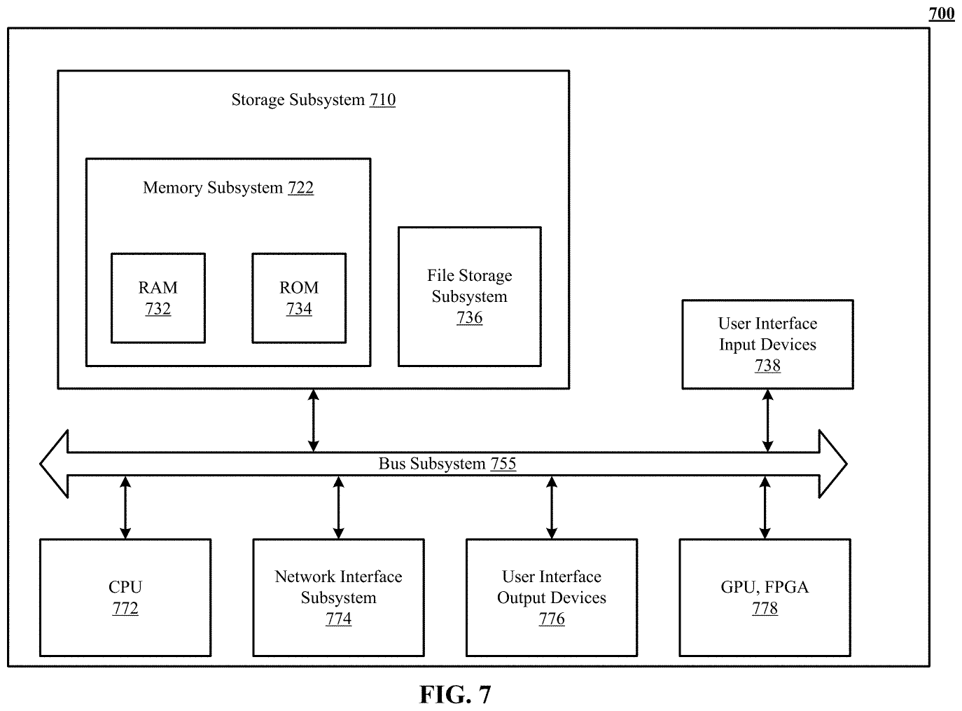

FIG. 7 is a simplified block diagram of a computer system 700 that can be used to implement the technology disclosed. Computer system typically includes at least one processor 772 that communicates with a number of peripheral devices via bus subsystem 755. These peripheral devices can include a storage subsystem 710 including, for example, memory subsystem 722 and a file storage subsystem 736, user interface input devices 738, user interface output devices 776, and a network interface subsystem 774. The input and output devices allow user interaction with computer system. Network interface subsystem provides an interface to outside networks, including an interface to corresponding interface devices in other computer systems.

User interface input devices 738 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

User interface output devices 776 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system to the user or to another machine or computer system.

Storage subsystem 710 stores programming and data constructs that provide the functionality of some or all of the modules and methods described herein. These software modules are generally executed by processor alone or in combination with other processors.

Memory used in the storage subsystem can include a number of memories including a main random access memory (RAM) 732 for storage of instructions and data during program execution and a read only memory (ROM) 734 in which fixed instructions are stored. The file storage subsystem 736 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem in the storage subsystem, or in other machines accessible by the processor.

Bus subsystem 755 provides a mechanism for letting the various components and subsystems of computer system communicate with each other as intended. Although bus subsystem is shown schematically as a single bus, alternative implementations of the bus subsystem can use multiple busses.

Computer system itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system depicted in FIG. 7 is intended only as a specific example for purposes of illustrating the technology disclosed. Many other configurations of computer system are possible having more or less components than the computer system depicted in FIG. 7.

The computer system 700 includes GPUs or FPGAs 778. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

### Particular Implementations

We disclose a system for remote medical information exchange in a variety of healthcare applications that can process facial information (such as image) or full body information of a user and produce inference information about the user.

One disclosed implementation includes a system for remote medical information exchange. The system includes a mirror, configured to capture the facial information of a user and display inference information about the user. The system includes an image capture module, configured to capture facial information of the user while the user is looking into the mirror. The system includes a displaying module, coupled to a display integrated into the mirror and configured to cause display of inference information about the user. The system includes an on-mirror computation device, coupled in communication with the image capture module and the displaying module, configured to receive and process facial information of the user and produce inference information about the user. The on-mirror computation device can comprise one or more artificial intelligence modules.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

The on-mirror computation device can further comprise a communication module, configured to communicate with a cloud-based server via an internet connection and exchange inference information about the user regarding a current and/or past sessions of the user looking into the mirror.

In one implementation, the mirror can comprise a video display that reflects to the user a video image captured by the image capture module. In another implementation, the mirror comprises a video display insert that displays the inference information to the user while a mirrored surface of the mirror reflects the user's reflection. The inference information displayed can include age of the user inferred from one or more captures of the facial information of the user. The inference information displayed can include height of the user inferred from one or more captures of the facial information of the user. The inference information displayed can include weight of the user inferred from one or more captures of the facial information of the user. The system can also display other inference information about the user, for example, sex, auto inference of mood and sentiments, auto inference of recommended health screenings and diagnostic information. The system can also display various health related measurements such as Body Mass Index (BMI), etc.

Other implementations may include a non-transitory computer readable storage medium storing instructions executable by a processor to perform actions of the system described above. Each of the features discussed in the particular implementation section for other implementations apply equally to this implementation. As indicated above, all the other features are not repeated here and should be considered repeated by reference.

A method implementation for remote medical information exchange via a mirror configured to capture the facial information of a user and display inference information about the user includes capturing facial information of the user while the user is looking into the mirror using an image capture module integrated into the mirror. The method includes causing display of the inference information about the user using a displaying module of the mirror, coupled to a display integrated into the mirror. The method includes receiving facial information of the user at an on-mirror computation device, coupled in communication with the image capture module and the displaying module. Finally, the method includes processing the facial information and producing the inference information about the user using one or more artificial intelligence modules.

Features described above for the system and described through out the application for systems and methods can be combined with this method, cast as it is from the server's perspective. In the interest of conciseness, not every combination of features is enumerated.

While the technology disclosed is disclosed by reference to the preferred embodiments and examples detailed above, it is to be understood that these examples are intended in an illustrative rather than in a limiting sense. It is contemplated that modifications and combinations will readily occur to those skilled in the art, which modifications and combinations will be within the spirit of the innovation and the scope of the following claims.

We claim as follows:

1. A system for remote medical information exchange, comprising:
a mirror, configured to capture facial information of a user and display inference information about the user, comprising
an image capture module, configured to capture facial information of the user while the user is looking into the mirror; and
a displaying module, coupled to a display integrated into the mirror and configured to cause display of inference information about the user; and
an on-mirror computation device, coupled in communication with the image capture module and the displaying module, configured to receive and process facial information of the user and produce inference information about the user, comprising one or more artificial intelligence modules.

2. The system of claim 1, wherein the on-mirror computation device further comprising a communication module, configured to communicate with a cloud-based server via an internet connection and exchange inference information about the user regarding a current and/or past sessions of the user looking into the mirror.

3. The system of claim 1, wherein the mirror comprises a video display that reflects to the user a video image captured by the image capture module.

4. The system of claim 1, wherein the mirror comprises a video display insert that displays the inference information to the user while a mirrored surface of the mirror reflects the user's reflection.

5. The system of claim 1, wherein the inference information displayed includes age of the user inferred from one or more captures of the facial information of the user.

6. The system of claim 1, wherein the inference information displayed includes height of the user inferred from one or more captures of the facial information of the user.

7. The system of claim 1, wherein the inference information displayed includes weight of the user inferred from one or more captures of the facial information of the user.

8. A method for remote medical information exchange via a mirror configured to capture facial information of a user and display inference information about the user, the method including:
capturing facial information of the user while the user is looking into the mirror using an image capture module integrated into the mirror; and
causing display of the inference information about the user using a displaying module of the mirror, coupled to a display integrated into the mirror; and
receiving facial information of the user at an on-mirror computation device, coupled in communication with the image capture module and the displaying module, and processing the facial information and produce the inference information about the user using one or more artificial intelligence modules.

9. The method of claim 8, further including using a communication module to exchange with a cloud-based server via an internet connection inference information about the user regarding a current and/or past sessions of the user looking into the mirror.

10. The method of claim 8, wherein the mirror substantially consists of a video display, further including reflecting to the user a video image captured by the image capture module.

11. The method of claim 8, wherein the mirror comprises a video display insert, further including causing display of the inference information to the user on the video display insert while a mirrored surface of the mirror reflects the user's reflection.

12. The method of claim 8, wherein the inference information displayed includes age of the user inferred from one or more captures of the facial information of the user.

13. The method of claim 8, wherein the inference information displayed includes height of the user inferred from one or more captures of the facial information of the user.

14. The method of claim 8, wherein the inference information displayed includes weight of the user inferred from one or more captures of the facial information of the user.

15. A non-transitory computer readable storage medium impressed with computer program instructions to implement remote medical information exchange via a mirror configured to capture facial information of a user and display inference information about the user, the instructions when executed on a processor, implement a method comprising:
capturing facial information of the user while the user is looking into the mirror using an image capture module integrated into the mirror; and
causing display of the inference information about the user using a displaying module of the mirror, coupled to a display integrated into the mirror; and
receiving facial information of the user at an on-mirror computation device, coupled in communication with the image capture module and the displaying module, and processing the facial information and produce the inference.

16. The non-transitory computer readable storage medium of claim 15, implementing the method further comprising, using a communication module to exchange with a cloud-based server via an internet connection inference information about the user regarding a current and/or past sessions of the user looking into the mirror.

17. The non-transitory computer readable storage medium of claim 15, wherein the mirror substantially consists of a video display, further including reflecting to the user a video image captured by the image capture module.

18. The non-transitory computer readable storage medium of claim 15, wherein the mirror comprises a video display insert, further including causing display of the inference information to the user on the video display insert while a mirrored surface of the mirror reflects the user's reflection.

19. The non-transitory computer readable storage medium of claim 15, wherein the inference information displayed includes age of the user inferred from one or more captures of the facial information of the user.

20. The non-transitory computer readable storage medium of claim 15, wherein the inference information displayed includes height of the user inferred from one or more captures of the facial information of the user.

21. The non-transitory computer readable storage medium of claim 15, wherein the inference information displayed includes weight of the user inferred from one or more captures of the facial information of the user.
